(19) United States
(12) Patent Application Publication
(10) Pub. No.: US 2024/0390451 A1
(43) Pub. Date: Nov. 28, 2024

(54) MODIFIED TRIPEPTIDES FOR USE IN THE TREATMENT OF A NON-ENVELOPED VIRUS INFECTION

(71) Applicant: PHARMA HOLDINGS AS, Tromsø (NO)

(72) Inventors: Christian LÜTKEN, Oslo (NO); Johnny Ivar RYVOLL, Tromsø (NO); Torsteinn ERLINGSSON, Tromsø (NO)

(21) Appl. No.: 18/697,318

(22) PCT Filed: Sep. 21, 2022
(86) PCT No.: PCT/EP2022/076271

§ 371 Date: Mar. 29, 2024

(30) Foreign Application Priority Data
Oct. 25, 2021 (GB) 2115324.2
Mar. 18, 2022 (GB) 2203804.6

(51) Int. Cl. A61K 38/06; A61P 31/16; C07K 5/09

(52) U.S. Cl. CPC A61K 38/06; A61P 31/16; C07K 5/0817

(57) ABSTRACT

The invention described herein relates to modified tripeptides, in particular LTX-109, for use in the treatment of non-enveloped virus infections.

The invention relates generally to treatment of certain viral infections. More particularly, the invention relates to the use of certain compounds for the treatment of non-enveloped virus infections.

Viruses are infectious agents that can only replicate within host organisms. Viruses can infect a variety of living organisms, including humans. Virus particles, when independent from their host cells, typically comprise a viral genome (which may be DNA or RNA, single- or double-stranded, linear or circular) contained within a protein shell called a capsid. In some viruses, termed enveloped viruses, the protein shell is enclosed in a membrane called an envelope. Other viruses are non-enveloped and so the capsid is the outermost part. Non-enveloped viruses are sometimes referred to as “naked” viruses.

Viral infections represent a significant healthcare problem. For example, at least 50% of those presenting with common cold symptoms have a causative rhinovirus infection, making rhinovirus one of the most prevalent and significant non-enveloped viruses in humans. There is an enormous societal cost associated with common colds in terms of missed school and work. Rhinovirus infection is also implicated in the more serious conditions of childhood otitis and childhood asthma exacerbations and in sinusitis. Other diseases caused by non-enveloped viruses include polio, aseptic meningitis, papillomas (warts) and acute infantile diarrhoea (winter diarrhoea; rotavirus)

Non-enveloped viruses lack the fragile lipid envelope and so may be more resistant to some disinfectants and other measures (pH and temperature) which may be used to suppress viruses. This may also explain the smaller number of agents available to treat non-enveloped virus infections than enveloped viral infections.

Nonetheless, there is great interest in effective anti-non-enveloped viral agents. For example, 25-hydroxycholesterol (25HC) has been shown to have marked antiviral activity against three pathogenic non-enveloped viruses, i.e. human papillomavirus-16 (HPV-16), human rotavirus (HRoV), and human rhinovirus (HRhV). Interferon-alpha has been shown to be effective against human rhinovirus infections but side effects and the development by volunteers of tolerance led to research into this treatment being abandoned, Pleconaril is a drug that prevents rhinoviruses from attaching to the host cell, but resistance causing mutations in the capsid protein (VP1) to which the drug binds can occur and reduce effectiveness. Viral resistance to antiviral agents is a significant problem in global health care

It is clear that alternative, and preferably advantageous, antiviral treatments (particularly viruses which cause disease in humans) would be highly desirable. Such treatments would be useful in treating or preventing infections by viral pathogens (e.g. in humans).

The present inventors have surprisingly found that a class of tripeptide compounds that carry a certain C-terminal modification exhibit excellent antiviral activity against non-enveloped viruses, including against non-enveloped viruses that are pathogenic to humans. Such tripeptides are cationic (positively charged) and bulky. One compound in this class is the compound LTX-109. LTX-109 has previously been reported to exhibit antibacterial activity (e.g. Saravolatz et al., Antimicrobial Agents and Chemotherapy (2012), Vol. 56(8) pages 4478-4482), but antiviral activity of these molecules has not previously been demonstrated. Given the findings of the present inventors, such compounds clearly represent an important class of agent to be added to the current arsenal of anti-non-enveloped virus therapies.

Thus, in one aspect, the present invention provides a compound for use in the treatment of a non-enveloped virus infection in a subject, wherein said compound is a compound of Formula (I)

AA-AA-AA-X—Y—Z (I)
wherein, in any order, 2 of said AA (amino acid) moieties are cationic amino acids, preferably lysine or arginine but may be histidine or any non-genetically coded or modified amino acid carrying a positive charge at pH 7.0, and 1 of said AA is an amino acid with a large lipophilic R group, the R group having 14-27 non-hydrogen atoms and preferably containing 2 or more, e.g. 2 or 3, cyclic groups which may be fused or connected, these cyclic groups will typically comprise 5 or 6 non-hydrogen atoms, preferably 6 non-hydrogen atoms (in the case of fused rings of course the non-hydrogen atoms may be shared);
X is a N atom, which may be, but preferably is not, substituted by a branched or unbranched C₁-C₁₀ alkyl or aryl group, e.g. methyl, ethyl or phenyl, and this group may incorporate up to 2 heteroatoms selected from N, O and S;
Y represents a group selected from —Rₐ—R_{b}—, —Rₐ—R_{b}—R_{b}— and —R_{b}—R_{b}—Rₐ
   wherein
Rₐ is C, O, S or N, preferably C, and
R_{b} is C; each of Rₐ and R_{b} may be substituted by C₁-C₄ alkyl groups or unsubstituted, preferably Y is —Rₐ—R_{b}— (in which Rₐ is preferably C) and preferably this group is not substituted, when Y is —Rₐ—R_{b}—R_{b}— or —R_{b}—R_{b}—Rₐ— then preferably one or more of Rₐ and R_{b} is substituted; and
Z is a group comprising 1 to 3 cyclic groups each of 5 or 6 non-hydrogen atoms (preferably C atoms), 2 or more of the cyclic groups may be fused; one or more of the rings may be substituted and these substitutions may, but will typically not, include polar groups, suitable substituting groups include halogens, preferably bromine or fluorine and C₁-C₄ alkyl groups; the Z moiety incorporates a maximum of 15 non-hydrogen atoms, preferably 5-12, most preferably it is phenyl;
the bond between Y and Z is a covalent bond between Rₐ or R_{b} of Y and a non-hydrogen atom of one of the cyclic groups of Z.

Suitable non-genetically coded amino acids and modified amino acids which can provide a cationic amino acid include analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

The large lipophilic R group of the AA may contain hetero atoms such as O, N or S, typically there is no more than one heteroatom, preferably it is nitrogen. This R group will preferably have no more than 2 polar groups, more preferably none or one, most preferably none.

Compounds for use in accordance with the invention are preferably peptides.

Compounds for use in accordance with the invention are preferably of formula (II):

AA₁-AA₂-AA₁-X—Y—Z (II)
wherein:
AA₁ is a cationic amino acid, preferably lysine or arginine but may be histidine or any non-genetically coded or modified amino acid carrying a positive charge at pH 7.0;
AA₂ is an amino acid with a large lipophilic R group, the R group having 14-27 non-hydrogen atoms and preferably containing 2 or more, e.g. 2 or 3, cyclic groups which may be fused or connected, these cyclic groups will typically comprise 5 or 6 non-hydrogen atoms, preferably 6 non-hydrogen atoms; and
X, Y and Z are as defined above.

Further preferred compounds for use in accordance with the invention include compounds of formulae (III) and (IV):

AA₂-AA₁-AA₁-X—Y—Z (III)

AA₁-AA₁-AA₂-X—Y—Z (IV)
wherein AA₁, AA₂, X, Y and Z are as defined above. Molecules of formula (II) are more preferred.

From amongst the above compounds certain are particularly preferred. In particular, compounds wherein the amino acid with a large lipophilic R group, conveniently referred to herein as AA₂, is tributyl tryptophan (Tbt) or a biphenylalanine derivative such as Phe(4-(2-Naphthyl)), Phe(4-(1-Naphthyl)), Bip (4-n-Bu), Bip (4-Ph) or Bip (4-T-Bu); Phe(4-(2-Naphthyl)), Phe(4-(1-Naphthyl)) and Tbt being most preferred. In some preferred embodiments, the amino acid with a lipophilic R group is tributyl tryptophan (Tbt).

In some preferred embodiments, Y is —Rₐ—R_{b}— and unsubstituted, most preferably Rₐ and R_{b} are both carbon (C) atoms. Preferably, Y is —CH₂—CH₂—.

In some preferred embodiments, Z is phenyl (Ph).

A further preferred group of compounds are those in which —X—Y—Z together is the group —NHCH₂CH₂Ph.

The compounds include all enantiomeric forms, both D and L amino acids and enantiomers resulting from chiral centers within the amino acid R groups and the C-terminal capping group “—X—Y—Z”. β and γ amino acids as well as a amino acids are included within the term ‘amino acids’, as are N-substituted glycines which may all be considered AA units. The compounds for use in accordance with the invention include beta peptides and depsipeptides.

The most preferred compound has the structural formula:

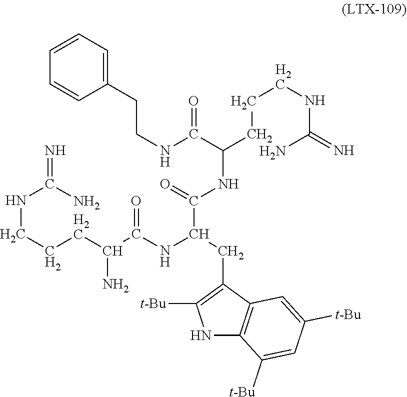 [SMILES: CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

t-Bu represents a tertiary butyl group. This compound with the structural formula above incorporating the amino acid 2,5,7-Tris-tert-butyl-L-tryptophan (this amino acid may also be referred to as tributyl tryptophan (Tbt)) is the most preferred compound for use in the present invention (and is also referred to herein as LTX-109). Analogues of this compound incorporating other cationic residues in place of Arg, in particular Lys, are also highly preferred. In one embodiment, one of the Arg residues in LTX-109 is substituted by a Lys residue, such as the N-terminal Arg or the C-terminal Arg. In another embodiment, both Arg residues in LTX-109 are substituted by Lys residues. In one embodiment, one of the Arg residues in LTX-109 is substituted by a His residue, such as the N-terminal Arg or the C-terminal Arg. In another embodiment, both Arg residues in LTX-109 are substituted by His residues.

Other cationic residues in place of Arg include suitable non-genetically coded amino acids and modified amino acids, including analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

Analogues incorporating alternative C terminal capping groups as defined above are also highly preferred.

Another preferred compound for use in accordance with the present invention is:

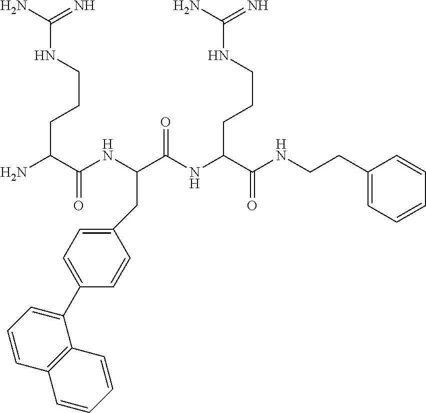 [SMILES: N=C(N)NCCCC(N)C(=O)NC(Cc1ccc(-c2cccc3ccccc23)cc1)C(=O)NC(CCCNC(=N)N)C(=O)NCCc1ccccc1]

This compound (i.e. the compound with the structural formula depicted immediately above) may be referred to as Arg-Phe(4-(1-Naphthyl))-Arg-NH—CH₂—CH₂-Ph. This compound is a compound of formula (II) in which AA₁ is arginine (Arg), AA₂ is Phe(4-(1-Naphthyl)), and —X—Y—Z together is the group —NHCH₂CH₂Ph.

Another preferred compound for use in accordance with the present invention is:

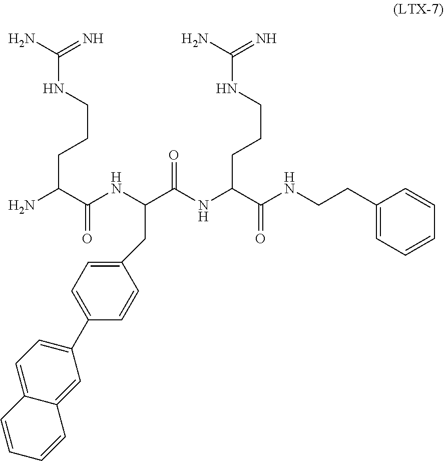 [SMILES: N=C(N)NCCCC(N)C(=O)NC(Cc1ccc(-c2ccc3ccccc3c2)cc1)C(=O)NC(CCCNC(=N)N)C(=O)NCCc1ccccc1]

This compound (i.e. the compound with the structural formula depicted immediately above) may be referred to as Arg-Phe(4-(2-Naphthyl))-Arg-NH—CH₂—CH₂-Ph. This compound is also referred to herein as LTX-7. This compound is a compound of formula (II) in which AA₁ is arginine (Arg), AA₂ is Phe(4-(2-Naphthyl)), and —X—Y—Z together is the group —NHCH₂CH₂Ph.

In some preferred embodiments, the compound for use in accordance with the present invention is LTX-109 or LTX-7. The compound LTX-109 is the most preferred compound for use in accordance with the present invention.

Compounds for use in the present invention are preferably peptides.

The compounds of formulae (I) to (IV) may be peptidomimetics and peptidomimetics of the peptides described and defined herein also represent compounds of use in accordance with the present invention. A peptidomimetic is typically characterised by retaining the polarity, three dimensional size and functionality (bioactivity) of its peptide equivalent but wherein the peptide bonds have been replaced, often by more stable linkages. By ‘stable’ is meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. Chapter 14 of “Drug Design and Development”, Krogsgaard, Larsen, Liljefors and Madsen (Eds) 1996, Horwood Acad. Pub provides a general discussion of techniques for the design and synthesis of peptidomimetics. In the present case, where the molecule may be reacting with a membrane rather than the specific active site of an enzyme, some of the problems described of exactly mimicking affinity and efficacy or substrate function are not relevant and a peptidomimetic can be readily prepared based on a given peptide structure or a motif of required functional groups. Suitable amide bond surrogates include the following groups: N-alkylation (Schmidt, R. et al., Int. J. Peptide Protein Res., 1995, 46, 47), retro-inverse amide (Chorev, M and Goodman, M., Acc. Chem. Res, 1993, 26, 266), thioamide (Sherman D. B. and Spatola, A. F. J. Am. Chem. Soc., 1990, 112, 433), thioester, phosphonate, ketomethylene (Hoffman, R. V. and Kim, H. O. J. Org. Chem., 1995, 60, 5107), hydroxymethylene, fluorovinyl (Allmendinger, T. et al., Tetrahydron Lett., 1990, 31, 7297), vinyl, methyleneamino (Sasaki, Y and Abe, J. Chem. Pharm. Bull. 1997 45, 13), methylenethio (Spatola, A. F., Methods Neurosci, 1993, 13, 19), alkane (Lavielle, S. et. al., Int. J. Peptide Protein Res., 1993, 42, 270) and sulfonamido (Luisi, G. et al. Tetrahedron Lett. 1993, 34, 2391).

The peptidomimetic compounds of use in the present invention will typically have 3 identifiable sub-units which are approximately equivalent in size and function to amino acids (AA units). The term ‘amino acid’ may thus conveniently be used herein to refer to the equivalent sub-unit of a peptidomimetic compound. Moreover, peptidomimetics may have groups equivalent to the R groups of amino acids and discussion herein of suitable R groups and of N and C terminal modifying groups applies, mutatis mutandis, to peptidomimetic compounds.

As is discussed in the text book referenced above, as well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Peptidomimetics and thus peptidomimetic backbones wherein the amide bonds have been replaced as discussed above are, however, preferred.

Suitable peptidomimetics include reduced peptides where the amide bond has been reduced to a methylene amine by treatment with a reducing agent e.g. borane or a hydride reagent such as lithium aluminium-hydride. Such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142. Strongly basic conditions will favour N-methylation over O-methylation and result in methylation of some or all of the nitrogen atoms in the peptide bonds and the N-terminal nitrogen.

Preferred peptidomimetic backbones include polyesters, polyamines and derivatives thereof as well as substituted alkanes and alkenes. The peptidomimetics will preferably have N and C termini which may be modified as discussed herein.

The compounds for use in the invention may be synthesised in any convenient way. Generally the reactive groups present (for example amino, thiol and/or carboxyl) will be protected during overall synthesis. The final step in the synthesis will thus be the deprotection of a protected derivative of the invention.

In building up a peptide, one can in principle start either at the C-terminal or the N-terminal although the C-terminal starting procedure is preferred.

Methods of peptide synthesis are well known in the art but for the present invention it may be particularly convenient to carry out the synthesis on a solid phase support, such supports being well known in the art.

A wide choice of protecting groups for amino acids are known and suitable amine protecting groups may include carbobenzoxy (also designated Z) t-butoxycarbonyl (also designated Boc), 4-methoxy-2,3,6-trimethylbenzene sulphonyl (Mtr) and 9-fluorenylmethoxy-carbonyl (also designated Fmoc). It will be appreciated that when the peptide is built up from the C-terminal end, an amine-protecting group will be present on the α-amino group of each new residue added and will need to be removed selectively prior to the next coupling step.

Carboxyl protecting groups which may, for example be employed include readily cleaved ester groups such as benzyl (Bzl), p-nitrobenzyl (ONb), pentachlorophenyl (OPCIP), pentafluorophenyl (OPfp) or t-butyl (OtBu) groups as well as the coupling groups on solid supports, for example methyl groups linked to polystyrene.

Thiol protecting groups include p-methoxybenzyl (Mob), trityl (Trt) and acetamidomethyl (Acm).

A wide range of procedures exists for removing amine- and carboxyl-protecting groups. These must, however, be consistent with the synthetic strategy employed.

The side chain protecting groups must be stable to the conditions used to remove the temporary α-amino protecting group prior to the next coupling step.

Amine protecting groups such as Boc and carboxyl protecting groups such as tBu may be removed simultaneously by acid treatment, for example with trifluoroacetic acid. Thiol protecting groups such as Trt may be removed selectively using an oxidation agent such as iodine.

Compounds for use in accordance with the present invention (e.g. LTX-109) may be synthesized as described in WO 2009/081152A2.

Compounds (e.g. peptides) for use in accordance with the present invention exhibit activity against non-enveloped viruses. Put another way, compounds for use in accordance with the present invention exhibit anti-non-enveloped virus activity.

Compounds of use in the present invention typically exhibit activity against non-enveloped viruses (anti-non-enveloped virus activity) in (or as determined by or as assessed by) a suitable in vitro assay, for example an endpoint dilution assay (e.g. a TCID50 assay). The skilled person is familiar with suitable in vitro assays, for example suitable endpoint dilution assays (e.g. TCID50 assays). Preferred TCID50 assays are described in the Example section herein.

Without wishing to be bound by theory, it is believed that the compounds of use in the present invention do not target a specific protein but instead the mechanism of action is more general. In particular, it is believed that there is an electrostatic mechanism whereby the positively charged compounds of the invention are attracted to negatively charged regions on the surface of non-enveloped viruses. This is advantageous both in terms of the breadth of viruses which can be treated and avoiding the development of resistance through specific mutations in the viral proteins.

As indicated above, the present invention provides compounds as defined elsewhere herein for use in treating non-enveloped virus infections. Put another way, the present invention provides a compound as defined herein for use in treating an infection in a subject, wherein the causative agent of said infection is a non-enveloped virus.

“Non-enveloped viruses” are viruses that lack a lipid layer (or lipid membrane). Thus, non-enveloped viruses have a capsid (viral protein capsid) as their outermost layer. The capsid shell surrounds the viral genome.

According to the present invention, preferred target viruses are icosahedral in their capsid morphology (these viruses are known as icosahedral viruses). While a variety of different sizes and arrangements of capsid proteins exist, these icosahedral viruses all have 20 triangular faces, made up of capsid proteins, which form an approximately spherical shape.

Any non-enveloped virus infection may be treated in accordance with the present invention. Typically and preferably, the non-enveloped virus is a virus that infects (or is capable of infecting) a mammal. Mammals include, for example, humans and any livestock, domestic or laboratory animal. Specific examples include mice, rats, pigs, cats, dogs, sheep, rabbits, cows and monkeys. In some embodiments of the present invention the mammal is a human. Thus, typically and preferably, the non-enveloped virus in accordance with the present invention is mammalian pathogen, preferably a human pathogen.

In some embodiments, the non-enveloped virus is a causative agent of a respiratory tract infection, also known as a respiratory virus. The respiratory tract infection may be an infection of the upper and/or lower respiratory tract. Upper respiratory tract infections are a preferred target for treatment according to the present invention.

The non-enveloped virus may be a DNA virus or a RNA virus. In some preferred embodiments, the non-enveloped virus is an RNA virus (e.g. a single stranded (ss) RNA non-enveloped virus).

Preferred target viruses are members of the Picornaviridae, Calciviridae, Parvoviridae, Papovaviridae, Papillomaviridae and Reoviridae families, with Picornaviridae being particularly preferred.

Within Picornaviridae, the genus Enterovirus is a preferred target. Structurally, all Enteroviruses are small, at 15-30 nm. The capsids contain positive-sense single-stranded RNA (+ssRNA) of approximately 7400 nucleotides in length. The genome, instead of having an AUG-containing cap, has an internal ribosomal entry site (IRES), which allows for mRNA translation. Within the genus Enterovirus are found enteroviruses, Coxsackie viruses, rhinoviruses, echoviruses and polioviruses; these are preferred virus targets according to the present invention. These are causative agents for a wide variety of illnesses ranging from the common cold (which is sometimes also referred to as viral rhinitis) to poliomyelitis and aseptic meningitis. In humans, they are among the most common infectious agents worldwide. In some embodiments, the non-enveloped virus may be a virus of one of the following species: Enterovirus A-D and Rhinovirus A-C. Rhinoviruses (all serotypes) are particularly preferred.

In some embodiments, the virus from the genus Enterovirus may be enterovirus C (sometimes also referred to as enterovirus species C or a type C enterovirus) or enterovirus D (sometimes referred to as enterovirus species D or a type D enterovirus). In some embodiments, the enterovirus C may be enterovirus C104 (EV-C104), enterovirus C105 (EV-C105), enterovirus C109 (EV-C109), enterovirus C117 (EV-C117), or enterovirus C118 (EV-C118). In some embodiments, the enterovirus D may be enterovirus D68 (EV-D68). Enterovirus C can cause, for example, the common cold (viral rhinitis) and/or pneumonia. Enterovirus D (e.g. enterovirus D68) can cause, for example, pneumonia.

In some embodiments, the virus from the genus Enterovirus may be a Coxsackie virus. A Coxsackie virus may be a group A Coxsackie virus (e.g. Coxsackie virus A21, also known as CV-A21) or a group B Coxsackie virus. Coxsackie viruses can cause, for example, upper respiratory tract infections, e.g. common cold (viral rhinitis).

In some embodiments, the virus from the genus Enterovirus may be an echovirus virus. Echoviruses can cause, for example, upper respiratory tract infections, e.g. viral rhinitis. Upper respiratory tract infections caused by echovirus may occur in particular in children.

As indicated above, in some embodiments rhinoviruses are preferred viruses in accordance with the invention. In some embodiments, the rhinovirus may be Rhinovirus A (e.g., Human Rhinovirus 60), Rhinovirus B (e.g., Human Rhinovirus 14) and/or Rhinovirus C.

In some embodiments, the virus from the family Papillomaviridae is a human papilloma virus (HPV). Human papilloma viruses can cause, for example, skin or mucous membrane growths (warts). In some embodiments, the virus from the family Calciviridae is a norovirus. Noroviruses can cause, for example, gastroenteritis. Norovirus infection is typically characterized by non-bloody diarrhoea, vomiting, stomach pain, fever and/or headaches. Alternatively viewed, in one aspect the present invention provides a compound as defined herein for use in treating a disease or condition caused by a non-enveloped virus infection. Embodiments of other aspects of the invention described herein apply, mutatis mutandis, to this aspect of the invention.

Diseases or conditions to be treated include infections of the upper or lower respiratory tract including the common cold (a term of the art used to refer to the cluster of symptoms including/selected from, blocked or runny nose, sore throat, sneezing, cough, muscle aches, headache, sinus pain and lethargy), otitis, sinusitis, pneumonia, bronchopneumonia, aseptic meningitis, polio, epidemic myalgia, hand, foot and mouth disease, myocarditis, pericarditis, pneumonitis and cerebella ataxia. The common cold and symptoms thereof are particularly preferred target conditions (patients will typically present with at least two or three common cold symptoms).

In some embodiments, compounds (or formulations or compositions) according to the present invention are for use in treating, such as reducing the severity and/or frequency of symptoms, a subject with an upper or lower respiratory tract infection (e.g., the common cold). In such embodiments, the upper or lower respiratory tract infection may be caused by a non-enveloped virus infection (e.g., a rhinovirus). In some embodiments, the present invention is for use in treating, such as reducing the severity and/or frequency of, blocked or runny nose, sore throat, sneezing, cough, muscle aches, headache, sinus pain and/or lethargy.

Other diseases or conditions to be treated include HPV infections. Thus, warts (e.g. skin warts or genital warts) are examples of other conditions that may be treated in accordance with the present invention.

In some embodiments, compounds (or formulations or compositions) according to the present invention are for use in treating, such as reducing the severity and/or frequency of symptoms, a subject with an HPV infection. In some embodiments, the present invention is for use in treating, such as reducing the severity and/or frequency of, skin or mucous membrane growths (warts).

Other diseases or conditions to be treated include norovirus infections. Thus, gastroenteritis caused by norovirus is an example of a condition that may be treated in accordance with the present invention.

In some embodiments, compounds (or formulations or compositions) according to the present invention are for use in treating, such as reducing the severity and/or frequency of symptoms, a subject with a norovirus infection. In some embodiments, the present invention is for use in treating, such as reducing the severity and/or frequency of, diarrhoea (e.g., non-bloody diarrhoea), vomiting, stomach pain, fever and/or headaches.

Compounds for use in accordance with the invention are typically presented (or administered) in the form of a formulation or composition comprising one or more compounds in accordance with the invention in admixture with a suitable diluent, carrier and/or excipient. Suitable diluents, excipients and carriers are known to the skilled person. Thus, the invention provides a formulation (or composition) comprising a compound as defined herein for use in treating a non-enveloped virus infection. Typically and preferably of course, the formulation (or composition) is a pharmaceutical formulation (or pharmaceutical composition). Thus, preferably diluents, carriers and/or excipients are pharmaceutically acceptable diluents carriers and/or carriers.

The compositions for use according to the invention may be presented, for example, in a form suitable for oral, nasal, respiratory tract (e.g. upper respiratory tract), parenteral, intravenous, topical or rectal administration. The skilled person is readily able to select an appropriate form for administration, for example based on the type of (or location of the) infection to be treated.

The compounds (or formulations or compositions) for use in accordance with the invention may be administered orally, nasally, parenterally, intravenously, topically or rectally.

In some embodiments, the compounds (or formulations or compositions) of the present invention are for administration to the upper or lower respiratory tract. For example, the compositions and formulations for use in accordance with the present invention may be administered using, e.g., a microcatheter (e.g., an endoscope and microcatheter), an aerosolizer, a powder dispenser, a nebulizer or an inhaler. Aptly, in some embodiments, the compounds (or formulations or compositions) are administered as a finely divided powder or a liquid aerosol.

In some embodiments, the compounds (or formulations or compositions) of the present invention are for nasal administration. For example, the compositions and formulations for use in accordance with certain embodiments of the present invention may be administered using a nasal applicator. Aptly, in some embodiments, the compounds (or formulations or compositions) are administered to a subject in a nasal formulation (for example as a finely divided powder or liquid solution).

The compositions for use according to certain embodiments of the invention may be obtained by conventional procedures using conventional pharmaceutical excipients, well known in the art.

The compounds (or formulations or compositions) for use in accordance with the invention may be administered to the respiratory tract, e.g. the upper respiratory tract.

As used herein, the term “pharmaceutical” includes veterinary applications of the invention.

The compounds defined herein may be presented in the conventional pharmacological forms of administration, such as tablets, coated tablets, solutions, emulsions, liposomes, powders, capsules, suppositories or sustained release forms.

Conventional pharmaceutical excipients as well as the usual methods of production may be employed for the preparation of these forms.

Tablets may be produced, for example, by mixing the active ingredient or ingredients with known excipients, such as for example with diluents, such as calcium carbonate, calcium phosphate or lactose, disintegrants such as corn starch or alginic acid, binders such as starch or gelatin, lubricants such as magnesium stearate or talcum, and/or agents for obtaining sustained release, such as carboxypolymethylene, carboxymethyl cellulose, cellulose acetate phthalate, or polyvinylacetate.

The tablets may if desired consist of several layers. Coated tablets may be produced by coating cores, obtained in a similar manner to the tablets, with agents commonly used for tablet coatings, for example, polyvinyl pyrrolidone or shellac, gum arabic, talcum, titanium dioxide or sugar. In order to obtain sustained release or to avoid incompatibilities, the core may consist of several layers too. The tablet-coat may also consist of several layers in order to obtain sustained release, in which case the excipients mentioned above for tablets may be used.

Solutions (e.g. injection solutions) may, for example, be produced in the conventional manner, such as by the addition of preservation agents, such as p-hydroxybenzoates, or stabilizers, such as EDTA. The solutions may be filled into vials or ampoules.

Capsules containing one or several active ingredients may be produced, for example, by mixing the active ingredients with inert carriers, such as lactose or sorbitol, and filling the mixture into gelatin capsules.

Suitable suppositories may, for example, be produced by mixing the active ingredient or active ingredient combinations with the conventional carriers envisaged for this purpose, such as natural fats or polyethylene glycol or derivatives thereof.

Dosages may vary based on parameters such as the age, weight and sex of the subject. Appropriate dosages can be readily established by the skilled person. Appropriate dosage units can readily be prepared.

Treatments in accordance with the present invention may involve co-administration with one or more further active agent that is used in the treatment or prevention of non-enveloped virus infections (or conditions caused thereby). Speaking generally, the one or more further active agent may be administered to the subject substantially simultaneously with the compound in accordance with the invention; such as from a single pharmaceutical composition or from two pharmaceutical compositions administered closely together. Thus, in some embodiments, pharmaceutical compositions may additionally comprise one or more further active ingredients (e.g. one or more further antiviral compounds). Alternatively, one or more further active agent may be administered to the subject at a time sequential to the administration of a compound in accordance with the invention. “At a time sequential”, as used herein, means “staggered”, such that the one or more further agent is administered to the subject at a time distinct to the administration of the compound in accordance with the invention. Generally, the two agents would be administered at times effectively spaced apart to allow the two agents to exert their respective therapeutic effects, i.e., they are administered at “biologically effective time intervals”. The one or more further active agent may be administered to the subject at a biologically effective time prior to the compound in accordance with the invention, or at a biologically effective time subsequent to the compound in accordance with the invention.

The term “treatment” or “therapy” used herein includes therapeutic and preventative (or prophylactic) therapies. Thus, compounds for use in accordance with the invention may be for therapeutic or prophylactic uses. A “preventive (or prophylactic) treatment” is a treatment administered to a subject who does not (or not yet) display signs or symptoms of, or displays only early signs or symptoms of, a disease, such that treatment is administered for the purpose of preventing or decreasing the risk of developing the disease and/or symptoms associated with the disease. A prophylactic treatment functions a treatment that inhibits or reduces further development or enhancement of the disease and/or its associated symptoms. A “therapeutic treatment” is a treatment administered to a subject who displays symptoms or signs of a disease, in which treatment is administered to the subject for the purpose of diminishing or eliminating those signs or symptoms, such as reducing the severity and/or frequency of symptoms, or for the purpose of delaying or stopping disease progression.

Alternatively viewed, the present invention provides a method of treating a non-enveloped virus infection in a subject (or patient) which method comprises administering to a subject in need thereof a therapeutically or prophylactically effective amount of a compound as defined herein. Embodiments of the invention described herein in relation to other aspects of the invention apply, mutatis mutandis, to this aspect of the invention.

The present invention also provides a method of treating a disease or condition that is caused by (or characterized by) a non-enveloped virus infection, which method comprises administering to a patient in need thereof a therapeutically or prophylactically effective amount of a compound as defined herein. Embodiments of the invention described herein in relation to other aspects of the invention apply, mutatis mutandis, to this aspect of the invention.

An effective amount (e.g. therapeutically or prophylactically effective amount) will be determined based on the clinical assessment and can be readily monitored. An amount administered should typically be effective to kill or inactivate all or a proportion of the target non-enveloped viruses or to prevent or reduce their rate of reproduction or otherwise to lessen their harmful effect on the body. Administration may also be prophylactic. Such an effective amount may be administered in one administration, i.e. one dose, or in several administrations, i.e. repetitive doses, i.e. in a series of doses, e.g. over the course of several days, weeks or months.

Further alternatively viewed, the present invention provides the use of a compound as defined herein in the manufacture of a medicament for use in the treatment of a non-enveloped virus infection. Embodiments of the invention described herein in relation to other aspects of the invention apply, mutatis mutandis, to this aspect of the invention.

Further alternatively viewed, the present invention provides the use of a compound as defined herein in the manufacture of a medicament for use in the treatment of a disease or condition that is caused by (or characterized by) a non-enveloped virus infection. Embodiments of the invention described herein in relation to other aspects of the invention apply, mutatis mutandis, to this aspect of the invention.

Further alternatively viewed, the present invention provides the use of a compound as defined herein for the treatment of a non-enveloped virus infection. Embodiments of the invention described herein in relation to other aspects of the invention apply, mutatis mutandis, to this aspect of the invention.

Further alternatively viewed, the present invention provides the use of a compound as defined herein for the treatment of a disease or condition that is caused by (or characterized by) a non-enveloped virus infection. Embodiments of the invention described herein in relation to other aspects of the invention apply, mutatis mutandis, to this aspect of the invention.

The term “subject” or “patient” as used herein includes any mammal, for example humans and any livestock, domestic or laboratory animal. Specific examples include mice, rats, pigs, cats, dogs, sheep, rabbits, cows and monkeys. Preferably, however, the subject or patient is a human subject. Thus, subjects or patients treated in accordance with the present invention will preferably be humans.

In some embodiments, subjects in accordance with the present invention are subjects having a non-enveloped virus infection. In some embodiments, subjects in accordance with the present invention are subjects suspected of having a non-enveloped virus infection. In some embodiments, subjects in accordance with the present invention may be subjects at risk of developing (or at risk of contracting) a non-enveloped virus infection.

In some embodiments, subjects in accordance with the present invention are subjects having a disease or condition caused by a non-enveloped virus infection. In some embodiments, subjects in accordance with the present invention are subjects suspected of having a disease or condition caused by a non-enveloped virus infection. In some embodiments, subjects in accordance with the present invention may be subjects at risk of developing (or at risk of contracting) a disease or condition caused by a non-enveloped virus infection.

The invention also provides kits comprising one or more of the compounds or compositions in accordance with the invention for use in the methods and uses described herein. Preferably said kits comprise instructions for use in treating non-enveloped virus infections as described herein.

As used throughout the entire application, the terms “a” and “an” are used in the sense that they mean “at least one”, “at least a first”, “one or more” or “a plurality” of the referenced components or steps, except in instances wherein an upper limit is thereafter specifically stated.

In addition, where the terms “comprise”, “comprises”, “has” or “having”, or other equivalent terms are used herein, then in some more specific embodiments these terms include the term “consists of” or “consists essentially of”, or other equivalent terms.

The invention will now be further described with reference to the following non-limiting Examples.

### EXAMPLES

### Example 1: Antiviral Activity of 1% LTX-109 Against Rhinovirus (Rhinovirus 60)

### Aim

The aim of this study was to test the antiviral activity of 1% LTX-109 against Rhinovirus.

### Methods

The Rhinovirus used was from BEI Resources: Rhinovirus 60, 2268-CV37 (Catalogue No. NR-51447).

To test whether 1% LTX-109 (w/v) has antiviral activity against Rhinovirus, 2.24×10⁵ infectious units of Rhinovirus (40 μl) were incubated with four volumes of 1% LTX-109 dissolved in PBS (160 μl), alongside a PBS (Phosphate-buffered saline) control and a 0.25% SDS (Sodium dodecyl sulphate) positive control. The treatments were performed in triplicate.

After 1 hour, the incubation was stopped by adding an excess of media, and the liquid was filtered through a filter (Sartorius VivaSpin 6) to separate the virus in order to reduce cytotoxicity on the assay cells. The assay media was DMEM (Gibco 61965-026) supplemented with 2% FBS (Gibco 10500-064), 20 mM Hepes (Gibco 15630-056) and 1×p/s (Gibco 15070063).

Infectious virus was quantified through a serial dilution of the filtrate (a series of ten-fold dilutions) on monolayers of HeLa cells in microtitre plates (HeLa cells are human cells capable of displaying a cytopathic effect (CPE) upon viral infection). For each dilution of the virus in the dilution series, eight wells of the microtitre plate were tested (i.e. each dilution was applied to eight separate wells, each well containing a HeLa cell monolayer). Appropriate controls were also performed. Seven days after infection of the cells, virus titre was quantified by determining the dilution at which half of the cells (half of the wells at a given dilution) displayed virus-induced cytopathic effect (TCID50). The TCID50 (TCID50/ml) assay (Tissue Culture Infectious Dose 50 assay) is a type of endpoint dilution assay that is well known in the art and routinely used to quantitatively measure virus titres. TCID50/ml provides a measure of infectious units of virus/ml. “/ml” refers to /ml of the starting solution (i.e. neat/undiluted solution) mentioned above. A parallel test where the same procedure was carried out in the absence of virus was included to determine any residual cytotoxic effect of the formulation on the assay cells.

### Results

The results of the test for antiviral activity of 1% LTX-109 against Rhinovirus are summarized in Table 1 (below).

After incubation with the PBS control for 1 hour, an average of 4.3E+05 TCID50/ml of Rhinovirus was measured.

After 1 hour incubation with 1% LTX-109, an average of 4.8E+03 TCID50/ml was measured, which corresponds to a decrease in infectivity of 1.9 logs, as compared to the PBS control.

After filtration, cytotoxicity on the HeLa cells was observed (only) with the neat application of the 1% LTX-109 formulation (without virus), but without affecting the validity of the test.

TABLE 1
Average virus titres recovered after incubation with PBS,
1% LTX-109 or 0.25% SDS for 1 hour.

| | | | | | Per- |
|---|---|---|---|---|---|
| | Cyto- | | | Log | cent- |
| | toxicity | TCID50/ml ± SEM | Log | decrease | age |
| PBS | No | 4.34E+05 ± 1.28E+05 | 5.6 | | |
| | cytotoxicity | | | | |
| 1% LTX-109 | Neat | 4.80E+03 ± 8.21E+02 | 3.7 | 1.9 | 98.89 |
| SDS | Neat | 1.00E+02 | 2.0 | 3.6 | 99.98 |

### Conclusion

Based on the findings reported here, exposure of Rhinovirus to 1% LTX-109 for 1 hour in vitro caused a 1.9 log decrease in virus infectivity as compared to the PBS control, which corresponds to a ˜99% reduction as compared to the PBS control. These results show that LTX-109 has excellent antiviral activity against Rhinovirus (a non-enveloped virus).

SDS as a positive control provides a benchmark and confirms the suitability of the assay. Non-enveloped viruses are known to be susceptible to SDS and while the impact of SDS slightly exceeds that of LTX-109, the test peptide still performs well in comparison.

The cytotoxicity test shows that direct application of LTX-109 to the Hela cells is only cytotoxic before any serial dilutions are performed (i.e. with the neat formulation). Thus any residual peptide which may be associated with the virus after the filtration step is not responsible for the activity seen in the TCID50 assay.

### Example 2: Antiviral Activity of 3% LTX-109 Against Rhinovirus (Rhinovirus 60)

### Aim

The aim of this study was to test the antiviral activity of 3% LTX-109 against Rhinovirus.

### Methods

The Rhinovirus used was from BEI Resources: Rhinovirus 60, 2268-CV37 (Catalogue No. NR-51447).

To test whether 3% LTX-109 (w/v) has antiviral activity against Rhinovirus, 9×10⁵ infectious units of Rhinovirus (40 μl) were incubated with four volumes of 3% LTX-109 dissolved in PBS (160 μl), alongside a PBS (Phosphate-buffered saline) control and a 0.25% SDS (Sodium dodecyl sulphate) positive control. The treatments were performed in triplicate.

After 1 hour, the incubation was stopped by adding 50 μl of mixture to 5 ml of media. The assay media was DMEM (Gibco 61965-026) supplemented with 2% FBS (Gibco 10500-064), 20 mM Hepes (Gibco 15630-056) and 1× p/s (Gibco 15070063).

Infectious virus was quantified through a serial dilution (a series of ten-fold dilutions) on a monolayer of HeLa cells in microtitre plates (HeLa cells are human cells capable of displaying a cytopathic effect (CPE) upon viral infection). For each dilution of the virus in the dilution series, eight wells of the microtitre plate were tested (i.e. each dilution was applied to eight separate wells, each well containing a HeLa cell monolayer). Appropriate controls were also performed. Seven days after infection of the cells, virus titre was quantified by determining the dilution at which half of the cells (half of the cells at a given dilution) displayed virus-induced cytopathic effect (TCID50). The TCID50 (TCID50/ml) assay (Tissue Culture Infectious Dose 50 assay) is a type of endpoint dilution assay that is well known in the art and routinely used to quantitatively measure virus titres. TCID50/ml provides a measure of infectious units of virus/ml. “/ml” refers to /ml of the starting solution (i.e. neat/undiluted solution) mentioned above.

A parallel test where the same procedure was carried out in the absence of virus was included to determine any residual cytotoxic effect of the formulation on the assay cells.

### Results

The results of the test for antiviral activity of 3% LTX-109 against Rhinovirus are summarized in Table 2 (below).

After incubation with the PBS control for 1 hour, an average of 1.76×10⁴ TCID50/ml of Rhinovirus was measured.

After 1 hour incubation with 3% LTX-109, an average of 1.99×10² TCID50/ml was measured, which corresponds to a decrease in infectivity of 1.9 logs, as compared to the PBS control.

After dilution, cytotoxicity on the HeLa cells was observed (only) with the neat application of the diluted 3% formulation (without virus), but without affecting the validity of the test.

TABLE 2
Average virus titres recovered after incubation with PBS,
3% LTX-109 or 0.25% SDS for 1 hour.

| | | | | | Per- |
|---|---|---|---|---|---|
| | Cyto- | | | Log | cent- |
| | toxicity | TCID50/ml ± SEM | Log | decrease | age |
| PBS | No | 1.76E+04 ± 5.63E+03 | 4.2 | | |
| | cytotoxicity | | | | |
| 3% LTX-109 | Neat | 1.99E+02 ± 4.11E+01 | 2.3 | 1.9 | 98.87 |
| SDS | Neat | 1.58E+01 | 1.2 | 3.0 | 99.91 |

### Conclusion

Based on the findings reported here, exposure of Rhinovirus to 3% LTX-109 for 1 hour in vitro caused a 1.9 log decrease in virus infectivity as compared to the PBS control, which corresponds to a ˜99% reduction as compared to the PBS control. These results show that LTX-109 has excellent antiviral activity against Rhinovirus (a non-enveloped virus).

SDS as a positive control provides a benchmark and confirms the suitability of the assay. Non-enveloped viruses are known to be susceptible to SDS and while the impact of SDS slightly exceeds that of LTX-109, the test peptide still performs well in comparison.

The cytotoxicity test shows that direct application of LTX-109 to the Hela cells is only cytotoxic before any serial dilutions are performed (i.e. with the neat liquid). Thus any residual peptide which may be associated with the virus is not responsible for the activity seen in the TCID50 assay.

### Example 3: Antiviral Activity of LTX-7 Against Rhinovirus (Rhinovirus 60)

### Aim

The aim of this study was to test the antiviral activity of LTX-7 against Rhinovirus.

### Methods

The Rhinovirus used was from BEI Resources: Rhinovirus (HRV-A60), Strain: 2268-CV37 (BEI Resources Catalogue Number NR-51447).

To test whether LTX-7 has antiviral activity against Rhinovirus, 5×10⁵ infectious units of Rhinovirus (40 μl) were incubated with four volumes of 1% LTX-7 (w/v) dissolved in PBS (160 μl) or a PBS (Phosphate-buffered saline) negative control. As a positive control, a buffer containing 0.25% SDS (Sodium dodecyl sulphate) in PBS was tested in parallel. Each sample and the PBS control were tested in triplicates.

After 1 hour at room temperature (RT), the incubation was stopped by adding an excess of cold assay media (5 ml), and the formulation was physically separated from the virus through a filter (Sartorius VivaSpin 6, 100,000 MWCO, PES (Sartorius, VS0642)) to reduce cytotoxicity on the assay cells. The assay media was DMEM (Gibco 61965-026) supplemented with 2% FBS (Gibco 10500-064), 20 mM Hepes (Gibco 15630-056) and 1× p/s (Gibco 15070063).

Infectious virus was quantified through a serial dilution (a series of ten-fold dilutions, 10⁰ to 10⁻⁷) on a monolayer of HeLaM cells plated in microtitre plates the day before at ˜7,000 cells/100 μl/well (HeLaM cells are human cells capable of displaying a cytopathic effect (CPE) upon viral infection). The starting solution for the serial dilution (i.e. the neat (or undiluted) solution or 10⁰ solution) was obtained by re-suspending the virus that was separated via the filtration step in 1 ml of assay media. For each dilution of the virus in the dilution series (10⁰ to 10⁻⁷), eight wells of the microtitre plate were tested (i.e. each dilution was applied to eight separate wells, each well containing a HeLaM cell monolayer). Appropriate controls were also performed. Seven days after infection of the cells, virus titre was quantified by determining the dilution at which half of the cells (half of the cells at a given dilution) displayed virus-induced cytopathic effect (TCID50), using the Reed and Muench method (L. J. Reed and H. Muench, American Journal of Epidemiology, Volume 27, Issue 3, 1938, Pages 493-497). The TCID50 (TCID50/ml) assay (Tissue Culture Infectious Dose 50 assay) is a type of endpoint dilution assay that is well known in the art and routinely used to quantitatively measure virus titres. TCID50/ml provides a measure of infectious units of virus/ml. “/ml” refers to /ml of the starting solution (i.e. neat/undiluted solution) mentioned above.

A parallel test where the same procedure was carried out in the absence of virus was included to determine any residual cytotoxic effect of LTX-7 on the assay cells.

### Results

The results of the test for antiviral activity of LTX-7 against Rhinovirus are summarized in Table 3 (below).

After incubation with the PBS control for 1 hour, an average of 9.17E+04 TCID50/ml of Rhinovirus was measured.

After 1 hour incubation with LTX-7, an average of 3.86E+03 TCID50/ml was measured, which corresponds to a decrease in infectivity of 1.33 logs, or at least 90%, as compared to the PBS control.

After 1 hour incubation with SDS (positive control), an average of 1.58E+01 TCID50/ml was measured, which corresponds to a decrease in infectivity of 3.67 logs, or ˜99.9%, as compared to the PBS control.

After filtration, cytotoxicity on the HeLaM cells was observed (only) with the neat application of the LTX-7 formulation and SDS (without virus), but without affecting the validity of the test.

TABLE 3
Average virus titres recovered after incubation with PBS or LTX-7. Virus titre
recovered after incubation with SDS is also shown.

| CONTACT | | | Log | % | Rounded |
|---|---|---|---|---|---|
| TIME: 1 hour | Cytotoxicity | TCID50/ml ± SEM | decrease | Reduction | % |
| PBS | No | 9.17E+04 ± 3.76E+04 | | | |
| | cytotoxicity | | | | |
| LTX-7 | Neat | 3.86E+03 ± 1.14E+03 | 1.33 | 95.791 | 90% |
| POSITIVE | Neat | 1.58E+01 (SEM ND) | 3.67 | 99.983 | 99.9% |
| CONTROL | | | | | |
| (SDS) | | | | | |

### Conclusions

Based on the findings reported here, exposure of Rhinovirus to LTX-7 for 1 hour in vitro caused a decrease of 1.33-logs in Rhinovirus infectivity, as compared to the PBS control. This corresponds to at least 90% reduction. These results show that LTX-7 has excellent antiviral activity against Rhinovirus (a non-enveloped virus).

SDS as a positive control provides a benchmark and confirms the suitability of the assay. Non-enveloped viruses are known to be susceptible to SDS and while the impact of SDS exceeds that of LTX-7, the test peptide (LTX-7) still performs well in comparison.

The cytotoxicity test shows that direct application of LTX-7 to the HeLaM cells is only cytotoxic before any serial dilutions are performed (i.e. with the neat formulation). Thus any residual peptide which may be associated with the virus after the filtration step is not responsible for the activity seen in the TCID50 assay.

### Example 4: Antiviral Activity of 1% LTX-109 Against Rhinovirus (Human Rhinovirus 14)

### Aim

The aim of this study was to test the antiviral activity of 1% LTX-109 against Human Rhinovirus 14.

### Method

The virus used was from ATCC: Human Rhinovirus 14, strain 1059 (ATCC VR-284, lot number 70049530).

To test whether 1% LTX-109 has virucidal activity against Human Rhinovirus 14, 4×10⁷ infectious units of Human Rhinovirus 14 in 40 μl were incubated with four volumes (160 μl) of 1% LTX-109 or a PBS negative control. As a positive control, a buffer containing 2.5% glutaraldehyde in PBS was tested in parallel. Both LTX-109 and negative control were tested in triplicates. The positive control was tested in a single replicate.

After 1 hour at room temperature (RT), the incubation was stopped by adding an excess of cold assay media (5 ml), and the formulation was physically separated from the virus through a filter (Sartorius VivaSpin 6, 100,000 MWCO, PES) to reduce cytotoxicity on the assay cells. The assay media was DMEM (Gibco 10566016) supplemented with 2% FBS (Gibco 10500064), 20 mM HEPES (Gibco 15630056) and 1× PenStrep (Gibco 15070063).

Concentrated virus was re-suspended in 1 ml of assay media and infectious virus was quantified through a serial dilution (10⁻¹ to 10⁻⁸) on a monolayer of HeLaM cells plated the day before at 8,000 cells/100 μl/well. For each dilution of the virus in the dilution series, four wells of the microtitre plate were tested (i.e. each dilution was applied to four separate wells, each well containing a HeLaM cell monolayer). Specifically, on a 96 well plate, 225 μl of media were placed in each well of 4 columns (32 wells in total). In the 4 wells of the top row (row A), 25 μl of re-suspended virus was added and mixed. With a multichannel pipette, 25 μl of media was then taken from the 4 wells in row A to the next 4 wells in row B, and then from row B to row C, and so on until row H, creating a serial dilution (10⁻¹ to 10⁻⁸). 200 μl from each well was then added to separate wells containing a monolayer of HeLa M cells. Appropriate controls were also performed. Three days after infection, virus titre was quantified by determining the dilution at which half of the cells displayed virus-induced cytopathic effect (TCID50), using the Reed and Muench method (L. J. Reed and H. Muench, American Journal of Epidemiology, Volume 27, Issue 3, 1938, Pages 493-497).

A parallel test where the same procedure was carried out in the absence of virus was included to determine any residual cytotoxic effect of LTX-109 on the assay cells.

### Results

The results of antiviral activity of 1% LTX-109 against Rhinovirus 14 are summarized in Table 4 (below).

After incubation with the PBS control for 1 hour, an average of 2.04E+07 TCID50/ml of Rhinovirus 14 was measured.

After 1 hour incubation with LTX-109, an average of 1.80E+06 TCID50/ml was measured, which corresponds to a decrease in infectivity of 1.05 logs, corresponding to 91.2% reduction in Rhinovirus 14 infectivity, as compared to the PBS control.

After 1 hour incubation with 2.5% glutaraldehyde in PBS (positive control), an average of 3.75E+01 TCID50/ml was measured, which corresponds to a decrease in infectivity of 5.74 logs, or ˜99.9%, as compared to the PBS control.

Cytotoxicity was observed for 1% LTX-109 when added to the assay cells at 10-1 dilution after filtration and re-suspension. No significant cytotoxicity was observed at greater dilutions. No significant cytotoxicity was observed for PBS.

TABLE 4
Average virus titres recovered after incubation with PBS or LTX-
109. Virus titre recovered after incubation with 2.5%
glutaraldehyde in PBS (positive control) is also shown.

| CONTACT | Cyto- | | Log | % |
|---|---|---|---|---|
| TIME: 1 hour | toxicity | TCID50/ml (±SEM) | Decrease | Reduction |
| PBS | No | 2.04E+07 (±8.55E+06) | — | — |
| | cyto- | | | |
| | toxicity | | | |
| 1% LTX-109 | 10⁻¹ | 1.80E+06 (±3.08 ± 05) | 1.05 | 91.17 |
| Positive | Neat | 3.75E+01 | 5.74 | 99.9998 |
| Control | | | | |

### Conclusions

Based on the findings reported here and under the conditions tested, exposure of Human Rhinovirus 14 to 1% LTX-109 for 1 hour caused a 1.05-log decrease in Human Rhinovirus 14 infectivity. This corresponds to 91.2% reduction.

Glutaraldehyde as a positive control provides a benchmark and confirms the suitability of the assay. Non-enveloped viruses are known to be susceptible to glutaraldehyde and while the impact of the positive control exceeds that of LTX-109, the test peptide (LTX-109) performs well in comparison.

Cytotoxicity was only observed for 1% LTX-109 when added to the assay cells at the 10-1 dilution after filtration and re-suspension. No significant cytotoxicity was observed at greater dilutions.

These results show that LTX-109 has excellent antiviral activity against Human Rhinovirus 14 (a non-enveloped virus).

1. A compound for use in the treatment of a non-enveloped virus infection in a subject, wherein said compound is a compound of Formula (I)
AA-AA-AA-X—Y—Z (1)
wherein, in any order, 2 of said AA (amino acid) moieties are cationic amino acids and 1 of said AA is an amino acid with a lipophilic R group, the R group having 14-27 non-hydrogen atoms;
X is a N atom, which may be substituted by a branched or unbranched C₁-C₁₀ alkyl or aryl group which group may incorporate up to 2 heteroatoms selected from N, O and S;
Y represents a group selected from —Rₐ—R_{b}—, —Rₐ—R_{b}—R_{b}— and —R_{b}—R_{b}—Rₐ— wherein
Rₐ is C, O, S or N, and
R_{b} is C; each of Rₐ and R_{b} may be substituted by C₁-C₄ alkyl groups or unsubstituted; and
Z is a group comprising 1 to 3 cyclic groups each of 5 or 6 non-hydrogen atoms, 2 or more of the cyclic groups may be fused and one or more of the cyclic groups may be substituted; the Z moiety incorporates a maximum of 15 non-hydrogen atoms; and
wherein
the bond between Y and Z is a covalent bond between Rₐ or R_{b} of Y and a non-hydrogen atom of one of the cyclic groups of Z.

2. The compound for use according to claim 1, wherein said compound is a peptide.

3. The compound for use according to claim 1 or claim 2, wherein said cationic amino acids are lysine and/or arginine.

4. The compound for use according to any one of claims 1 to 3, wherein said cationic amino acids are arginine.

5. The compound for use according to any one of claims 1 to 4, wherein the lipophilic R group contains 2 or more cyclic groups which may be fused or connected.

6. The compound for use according to any one of claims 1 to 5, wherein X is unsubstituted.

7. The compound for use according to any one of claims 1 to 6, wherein Rₐ is C.

8. The compound for use according to any one of claims 1 to 7, wherein Y is —Rₐ—R_{b}— and unsubstituted.

9. The compound for use according to any one of claims 1 to 8, wherein Y is —CH₂—CH₂—.

10. The compound for use according to any one of claims 1 to 9, wherein Z is phenyl.

11. The compound for use according to any one of claims 1 to 10, wherein said compound is a compound of formula (II)
AA₁-AA₂-AA₁-X—Y—Z (II)
wherein:
AA₁ is a cationic amino acid;
AA₂ is an amino acid with a lipophilic R group, the R group having 14-27 non-hydrogen atoms; and
X, Y and Z are as defined in any one of claims 1 to 10.

12. The compound for use according to any one of claims 1 to 11, wherein the amino acid with a lipophilic R group is selected from tributyl tryptophan (Tbt) or a biphenylalanine derivative selected from Phe (4-(2-Naphthyl)), Phe (4-(1-Naphthyl)), Bip (4-n-Bu), Bip (4-Ph) and Bip (4-T-Bu).

13. The compound for use according to any one of claims 1 to 12, wherein the amino acid with a lipophilic R group is tributyl tryptophan (Tbt).

14. The compound for use according to any one of claims 1 to 13, wherein —X—Y—Z together are —NHCH₂CH₂Ph.

15. The compound for use according to any one of claims 1 to 14, wherein said compound has the structural formula:

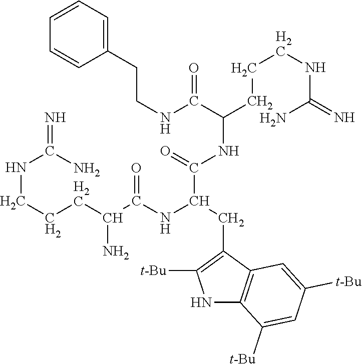 [SMILES: CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

16. The compound for use according to any one of claims 1 to 12 wherein the amino acid with a lipophilic R group is Phe (4-(2-Naphthyl)).

17. The compound for use according to any one of claims 1 to 12 or 16, wherein said compound has the structural formula:

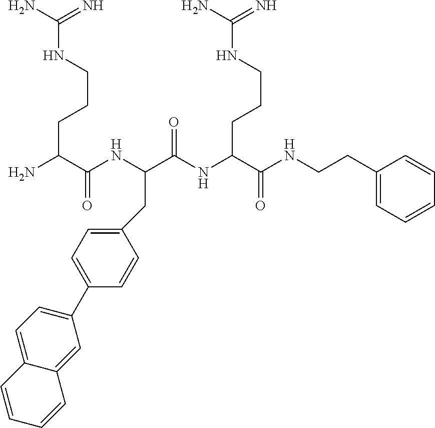 [SMILES: N=C(N)NCCCC(N)C(=O)NC(Cc1ccc(-c2ccc3ccccc3c2)cc1)C(=O)NC(CCCNC(=N)N)C(=O)NCCc1ccccc1]

18. The compound for use according to any one of claims 1 to 17, wherein said non-enveloped virus infection is a respiratory tract infection.

19. The compound for use according to any one of claims 1 to 18, wherein said non-enveloped virus infection is an upper respiratory tract infection.

20. The compound for use according to any one of claims 1 to 19, wherein said non-enveloped virus is a virus of the family Picornaviridae.

21. The compound for use according to any one of claims 1 to 20, wherein said non-enveloped virus is Rhinovirus A, Rhinovirus B or Rhinovirus C.

22. The compound of any one of claims 1 to 21, wherein said subject is a human subject.

23. A pharmaceutical formulation comprising a compound as defined in any one of claims 1 to 17 and a diluent, carrier and/or excipient for use in the treatment of a non-enveloped virus infection in a subject.

24. The compound for use according to any one of claims 1 to 22 or the pharmaceutical composition for use according to claim 23, wherein said treatment is administered using a microcatheter, an aerosolizer, a powder dispenser, a nebulizer, an inhaler and/or nasal applicator.

25. The compound for use according to any one of claims 1 to 22 or 24 or the pharmaceutical composition for use according to claim 23 or 24, wherein said treatment is a therapeutic treatment.

26. The compound for use according to any one of claims 1 to 22 or 24 or the pharmaceutical composition for use according to claim 23 or 24, wherein said treatment is a prophylactic treatment.

27. A method of treating a non-enveloped virus infection in a subject, which method comprises administering to a subject in need thereof an effective amount of a compound as defined in any one of claims 1 to 17.

28. Use of a compound as defined in any one of claims 1 to 17 in the manufacture of a medicament for use in the treatment of a non-enveloped virus infection.
